(19) United States
(12) Patent Application Publication
(10) Pub. No.: US 2006/0257473 A1
(43) Pub. Date: Nov. 16, 2006

(54) Extended release tablet

(76) Inventor: Porranee Puranajoti, Hoffman Estates, IL (US)

Correspondence Address: PRICE HENEVELD COOPER DEWITT & LITTON, LLP, 695 KENMOOR, S.E., P O BOX 2567, GRAND RAPIDS, MI, 49501, US

(21) Appl. No.: 11/399,203

(22) Filed: Apr. 6, 2006

Related U.S. Application Data
(60) Provisional application No. 60/679,838, filed on May 11, 2005.

(51) Int. Cl. A61K 9/20; A61K 9/14; A01N 31/14

(52) U.S. Cl. 424/464; 424/489; 514/718

(57) ABSTRACT

A single tablet layer having an extended release profile comparable to the release profile of a bi-layer tablet having both an immediate release and an extended release layer is prepared from a pharmaceutical granulation containing a pharmaceutically active compound, a hydrophilic polymer, and a water in-soluble, non-swellable particulate channeling agent.

### CROSS-REFERENCE TO RELATED APPLICATION

This application claims priority under 35 U.S.C. § 119(e) of U.S. Provisional Application No. 60/679,838 entitled EXTENDED RELEASE TABLET, filed May 11, 2005, the entire disclosure of which is incorporated herein by reference.

### FIELD OF THE INVENTION

This invention pertains to pharmaceutical dosage forms, and more particularly to tablets providing sustained release or a combination of both immediate and sustained release of a pharmaceutically active compound.

### BACKGROUND OF THE INVENTION

It is known in the pharmaceutical art that sustained release dosage forms often have advantages as compared with immediate release dosage forms for the administration of many therapeutically active compounds. These advantages include better patient compliance with a prescribed therapeutic regimen due to fewer dose administrations, and maintenance of a more constant, safe and therapeutically effective blood serum concentration, thereby providing a more consistent therapeutic effect over an extended duration of time. It is also recognized that sustained release dosage forms may employ less total drug to achieve a desired therapeutic effect, thereby minimizing undesirable side effects and providing improved economics.

Pharmaceutically active compounds are most frequently and most desirably administered orally by means of solid dosage forms, most typically tablets or capsules.

Conventional sustained release tablets usually comprise either a core containing a pharmaceutically active compound surrounded by a membrane, or a matrix material into which the pharmaceutically active compound is uniformly distributed in a finely divided form.

The membrane or matrix material is usually theoretically selected to provide a dosage form in which the release rate is controlled by either the rate at which the pharmaceutically active compound diffuses through a water-insoluble matrix or membrane material, or by the rate of dissolution of the matrix or membrane material. Although one mechanism may predominantly control the release rate, the actual overall release rate is usually dependent on both the rate of diffusion of the pharmaceutically active compound through the matrix or membrane, and the dissolution rate of the matrix or membrane.

It is also known to employ channeling agents or wicking agents to modify the release rate of a drug from a matrix material or through a membrane. Channeling agents are typically water-swellable and/or water-soluble materials (such as lactose) that are distributed in particle form in a matrix or membrane to provide tortuous channels that have the effect of increasing the surface area available for dissolution of the matrix or membrane and/or decreasing the thickness of the rate limiting diffusion barrier. Wicking agents are usually water-insoluble materials (such as microcrystalline cellulose) that utilize surface energy or capillary action to achieve an effect similar to that of a channeling agent.

In those cases where it is desirable to achieve both immediate and sustained release in a single tablet, a bi-layer tablet having separate immediate and sustained release portions is typically utilized. Bi-layer tablets are prepared by separately formulating the materials of the individual layers, employing steps such as granulating, milling and blending. A first layer is compressed, and, thereafter, the second layer is compressed adjacent to the first layer. Preparation of bi-layer and other multiple compressed layer tablets is difficult and expensive. Accordingly, it would be more economical to achieve a desired release rate and therapeutic effect using a tablet having a single compressed layer.

United States Patent Application Publication No. 2002/0051817 discloses a modified release composition for a pharmaceutically acceptable salt of diclofenac. The composition comprises about 5-25 percent by weight of hydroxyethyl cellulose, about 5-75 percent by weight of lactose, about 0-3 percent by weight of silicon dioxide, about 0.5-5 percent by weight of polyvinylpyrrolidone, less than 3 percent by weight of talc, and less than 3 percent by weight of magnesium stearate. This publication does not mention guaifenesin.

United States Patent Application Publication No. 2002/0044968 discloses use of a hydrophilic component in a controlled release dosage form in which the pharmaceutically active material, which for example may be guaifenesin, is embedded in a plasticized matrix material.

United States Patent Application Publication No. 2004/0022851 discloses a drug delivery system comprising a sustained release portion containing guaifenesin, pseudoephedrine, and a release-delaying matrix containing a hydrophilic polymer and a water-insoluble polymer; and an immediate release portion comprising guaifenesin. It is disclosed that colloidal silicon dioxide and/or talc may be utilized as extragranular excipients, namely as glidants.

U.S. Pat. No. 5,032,406 discloses a dual-action tablet comprising an outer tablet having a first dose of active ingredient dispersed in a pH independent hydrophilic polymer matrix and an inner tablet comprising a second dose of active ingredient in a rapidly disintegrating excipient base.

U.S. Pat. Nos. 6,372,252 and 6,955,821 describes a pharmaceutical sustained release formulation of guaifenesin comprising a hydrophilic polymer, such as hydroxypropyl methylcellulose, and a water-insoluble polymer, preferably an acrylic resin.

U.S. Pat. No. 5,445,829 discloses an extended release pharmaceutical dosage form adapted to approach zero order release of drug over a 12 to 24 hour period. The formulation comprises a mixture of zero to about 50 percent of an immediate release particle containing a drug, inert substrate and binder, coated with talc and up to 100% of an extended release particle comprising the immediate release particle coated with a dissolution modifying system containing plasticizers and a film-forming agent.

U.S. Pat. No. 6,150,410 discloses a pharmaceutical formulation of acidic pharmacologic agents. The composition comprises about 10 weight percent to about 40 weight percent of a pharmaceutically acceptable neutral, water-swellable, hydrophilic polymer, and from about 15 weight percent to about 50 weight percent of a pharmaceutically acceptable acid soluble polymer which is water-swellable above pH 5. Colloidal silicon dioxide may be used as a glidant (extragranular component) in an amount of about 1 percent by weight or less.

U.S. Pat. Nos. 6,531,152 and 6,632,451 disclose coatings having a water-insoluble hydrophilic particulate matter that forms channels that connect a core containing a pharmaceutically active compound with either an outer surface or an outer coating.

### SUMMARY OF THE INVENTION

The invention pertains to a single compressed tablet layer that in certain embodiments achieves a release profile of a pharmaceutically active compound having both immediate and extended release portions. In certain preferred embodiments, the dosage form of the invention can be formulated as a single tablet layer or portion that provides a combination of immediate and extended release that is equivalent to a tablet having separate immediate and extended release layers or portions. Using a single layer or portion, rather than two separate layers or portions, for achieving a combination of immediate and sustained release of a pharmaceutically active compound substantially reduces the manufacturing cost of the tablets by eliminating mixing, granulating and compressing steps for one of the two layers or portions.

In certain embodiments, the pharmaceutical granulation includes a pharmaceutically active compound, a hydrophilic polymer, a non-hygroscopic diluent and a water-insoluble, non-swellable particulate channeling agent in an amount of from 0.1 to 4.0 percent by weight.

In a specific embodiment of the invention, the pharmaceutical granulation comprises guaifenesin, a hydrophilic polymer, and a water-insoluble, non-swellable particulate channeling agent.

The pharmaceutical granulations of this invention may be compressed into a single tablet layer capable of providing a release rate of a pharmaceutically active compound that is comparable to a tablet having separate immediate and sustained release portions. The compressed layer may be utilized alone or in combination with additional layers containing different pharmaceutically active compounds, and may be provided with aesthetic and/or functional coatings, such as an enteric coating.

These and other features, advantages and objects of the present invention will be further understood and appreciated by those skilled in the art by reference to the following specification and claims.

### DESCRIPTION OF THE PREFERRED EMBODIMENTS

The invention involves the use of a water-insoluble, non-swellable channeling agent and a hydrophilic polymer in a granulation containing a pharmaceutically active compound to allow compression of the granulation into a single tablet layer or portion that in certain embodiments exhibits a combination of immediate and extended release equivalent or comparable to the release properties of a tablet having separate immediate and extended release portions. This combination of a hydrophilic polymer and a channeling agent allows an aqueous medium (e.g., gastric juices) to become quickly imbibed or absorbed by the tablet, swell, and form channels that allow a portion of the pharmaceutically active ingredient to be immediately released, while allowing another portion of the pharmaceutically active ingredient to be slowly released over an extended period of time.

The granulations of this invention may be used for sustained release, or a combination of immediate and sustained release, for a variety of pharmaceutically active compounds, regardless of their solubility in water, including compounds that are highly soluble in water, as well as those that are sparingly soluble in water. Examples of pharmaceutically active compounds that may be utilized include analgesics, such as acetaminophen, ibuprofen, flurbiprofen, ketoprofen, voltaren, phenacetin and salicylamide; anti-inflammatories such as naproxen and indomethacin; antihistamines, such as chlorpheniramine maleate, phenindamine tartrate, pyrilamine maleate, doxylamine succinate, phenyltoloxamine citrate, diphenhydramine hydrochloride, promethazine, brompheniramine maleate, dexbrompheniramine maleate, clemastine fumerate and triprolidine; antitussives such as dextromethorphan hydrobromide; expectorants such as guaifenesin; decongestants such as phenylephrine hydrochloride, phenylpropanolamine hydrochloride, pseudoephedrine hydrochloride, ephedrine; narcotics such as morphine and codeine; cardiovascular drugs such as diltiazem, propranolol, nifedepine and clonidine; central nervous system drugs such as thioridazine, diazepam, meclizine, ergoloid mesylates, chlorpromazine, carbidopa and levodopa. In addition, various combinations of pharmaceutically active compounds may be employed in one or two or more layers in accordance with the invention. The pharmaceutically active compounds are employed in amounts to provide a tablet layer or portion containing a therapeutically effective dose. A therapeutically effective dose is an amount that reproducibly provides an observable therapeutic benefit in a statistically significant sample of the population. Depending on the particular pharmaceutically active compound, a therapeutically effective amount may range from about 4 percent to about 90 percent of the weight of the granulation.

The hydrophilic polymers that may be employed include polymers that gel and dissolve slowly in an aqueous media. Examples of suitable hydrophilic polymers include acacia, gum tragacanth, locust bean gum, guar gum, karaya gum, methylcellulose, hydroxymethyl cellulose, hydroxypropyl methylcellulose, hydroxypropyl cellulose, hydroxyethyl cellulose, carboxymethylcellulose, agar, pectin, carrageen, alginates, and carboxymethylcellulose. A preferred hydrophilic polymer, such as for use with guaifenesin, is hydroxyethyl cellulose. The amount of hydrophilic polymer employed in the composition depends, among other things, on the particular pharmaceutically active compound or compounds employed, and the use and amount of any diluents. However, the compressed tablet layer typically comprises about 3 to about 30 percent hydrophilic polymer by weight.

Channeling agents employed in the granulations, tablet layers and tablets of this invention are water-insoluble, non-swellable particulate materials that are capable of absorbing or adsorbing and transporting moisture. A preferred channeling agent is silicon dioxide. A suitable and preferred commercially available silicon dioxide is SYLOID® 244, which is a high purity (99.6% SiO₂) micronized free flowing powder. The channeling agent, such as silicon dioxide, is typically employed in an amount of from 0.1 to 4.0 percent by weight of the granulation, and more typically 0.5 to 2.0 percent by weight of the granulation.

In addition to the pharmaceutically active compound, hydrophilic polymer and channeling agent, the pharmaceutical granulations of this invention typically include a polymeric binder. Preferred polymeric binders include water-soluble polymeric binders, such as copovidone, which is a linear, random copolymer of N-vinyl-2-pyrrolidone and vinyl acetate. The polymeric binder(s) are typically employed in an amount of from about 0.5 to 5 percent by weight of the granulation, although lower or higher amounts may be employed depending on the particular active(s) and desired release properties.

In addition to the water-insoluble, non-swellable channeling agents, water-soluble channeling agents, such as compressible sugar (e.g., a combination of maltose and sucrose), may be employed in the granulation in an amount of up to about 10 percent by weight of the granulation, and more typically up to 5 percent by weight of the granulation.

It has been determined that the release properties are influenced by the manner in which the granules are prepared, including the order in which the components are mixed. In order to achieve a desired release profile comparable to the release profile of a bi-layer tablet having both an immediate release layer and a sustained release layer, it is desirable to mix the pharmaceutically active compound with the channeling agent before adding the hydrophilic polymer, and optional polymeric binder. By mixing the channeling agent with the pharmaceutically active compound before adding the polymeric materials, immediate release of a portion of the pharmaceutically active compound is enhanced. It is believed that combining the pharmaceutically active compound with the channeling agent before adding the polymeric materials minimizes direct contact between the polymeric materials and the pharmaceutically active compound, and allows a portion of the pharmaceutically active material to dissolve and release quickly.

After the pharmaceutically active ingredient has been thoroughly mixed with the channeling agent, the hydrophilic polymer, and any polymeric binders and diluents can be added. Preferred diluents are non-hygroscopic to prevent excessive absorption and/or swelling upon contact of the dosage form with an aqueous medium. A preferred non-hygroscopic diluent is dicalcium phosphate anhydrous, which may be employed in amount of up to about 25 percent by weight of the granulation.

The granulation can be prepared, for example, by using a TK Fielder granulator by spraying purified water. Typically, the amount of water is about 5 to 40 percent of the weight of the ingredients being granulated. The granules are typically dried and milled. Desirably, the granules are dried to a moisture content of about 0.5-2.0 percent water by weight.

The resulting granulation is compressed into tablets, typically with the aid of additional binder such as copovidone, and a lubricant, such as zinc stearate. Extragranular ingredients may also include additional channeling agents that are either water-insoluble (e.g., silicon dioxide) or water-soluble (e.g., compressible sugar), colorants (e.g., pigments or dyes), and other conventional tableting aids. Extragranular channeling agents can be used in amounts up to about 10 percent of the table weight, more typically up to about 2.5 percent of the tablet weight.

It is conceivable that the concepts of the invention can be employed in multiple layer or portion tablets, such as a tablet having a compressed tablet layer or portion in accordance with the invention, and one or more additional layers or portions. These additional layers or portions can either be prepared using a granulation in accordance with the invention, or prepared using a compressible formulation different from the granulations of this invention. The single portion and multiple portion tablets may be provided with an aesthetic coating, or a functional coating, e.g., enteric and/or taste-masking coatings.

The invention is illustrated by the following examples.

### EXAMPLE 1

Guaifenesin tablets are prepared in accordance with the following formulation.

| Name of Ingredient | Mg/Tablet |
|---|---|
| Guaifenesin | 600 |
| SYLOID ® 244 (Silicon Dioxide) | 3 |
| Dicalcium Phosphate Anhydrous | 50 |
| NATROSOL ® 250 HHX (Hydroxyethyl Cellulose) | 108 |
| Copovidone | 30 |
| Zinc Stearate | 6 |

A premix granulation is prepared by first passing guaifenesin through a Fitzmill milling machine (Screen # 15310093) into a V-shell mixer. The SYLOID® 244 silicon dioxide is then added into the V-shell mixer and mixed with the guaifenesin for five minutes. Thereafter, the dicalcium phosphate anhydrous, NATROSOL® 250 HHX hydrophilic polymer (hydroxyethyl cellulose), and 80 percent of the total copovidone are added into the V-shell mixer and mixed for five minutes with the guaifenesin and silicon dioxide. The premix granulation is completed using a TK Fielder granulator and purified water for wet granulation. The resulting granules are passed through a Comil milling machine to obtain a wet granulation (Screen # 2F083R050/42, speed 300 rpm). The wet granulation is placed in a fluid bed dryer (Glatt WSG 5) and dried to a moisture content of from about 0.5-2.0 percent at 80° C. for 10 minutes, as determined by a Denver Moisture Tester. The dried granulation is milled in a Comil milling machine (Screen # 2F083R050/42, speed 300 rpm).

The resulting dried and milled granules are combined with the remaining copovidone in a V-shell mixer and mixed for five minutes. Thereafter, zinc stearate passed through a 30-mesh screen is added and mixed with the granulation and copovidone for about three minutes.

The resulting mixture is compressed on a Betapress tablet press to obtain a 797 mg tablet containing 600 mg of guaifenesin.

### EXAMPLE 2

A guaifenesin granulation comprising both intragranular (i.e., within the granule) and extragranular (i.e., outside of the granules) water-soluble and water-insoluble channeling agents is prepared in accordance with the following formulation.

GRANULATION

| Name of Ingredient | Mg/Tablet |
|---|---|
| Guaifenesin, USP | 600 |
| SYLOID ® 244 (Silicon Dioxide) | 12 |
| Dicalcium Phosphate Anhydrous | 12 |
| Compressible Sugar | 20 |
| NATROSOL ® 250 HHX (Hydroxyethyl Celluose) | 90 |
| Copovidone | 22 |

A premix is prepared by first passing guaifenesin through a milling machine into a V-shell mixer, adding the SYLOID® 244 silicon dixoxide to the V-shell mixer, and mixing the guaifenesin with the silicon dioxide for five minutes. Thereafter, the dicalcium phosphate anhydrous, NATROSOL® 250 HHX hydrophilic polymer (hydroxyethyl cellulose) and compressible sugar are added to the V-shell mixer and mixed for 10 minutes with the guaifenesin and silicon dioxide. The resulting premix is combined with an aqueous copovidone solution and granulated in a granulator to obtain a wet granulation. The wet granulation is placed in a fluid bed dryer and dried to a moisture content of about 0.5-2.0% at 80° C. for 10 minutes. The dried granulation is milled (in a Comil milling machine).

TABLET FORMULATION

| Name of Ingredient | Mg/Tablet |
|---|---|
| Guaifenesin granulation | 756 |
| SYLOID ® 244 (Silicon Dioxide) | 15 |
| Compressible Sugar | 30 |
| Zinc Stearate | 6 |

The guaifenesin granulation is transferred to a V-blender, and the additional SYLOID® 244 (silicon dioxide) and compressible sugar are added and mixed for 15 minutes. Thereafter, the zinc stearate is passed through a 30-mesh screen and added to the V-blender, and mixed with the granulation, and extragranular compressible sugar and silicon dioxide for five minutes.

The resulting mixture is compressed to obtain a 807 milligram tablet containing 600 milligrams of guaifenesin.

### EXAMPLE 3

A guaifenesin granulation comprising both intergranular and extragranular water-soluble and water-insoluble channeling agents is prepared in accordance with the following formulation.

GRANULATION

| Name of Ingredient | Mg/Tablet |
|---|---|
| Guaifenesin, USP | 600 |
| SYLOID ® 244 (Silicon Dioxide) | 10 |
| Dicalcium Phosphate Anhydrous | 3 |
| Compressible Sugar | 25 |
| NATROSOL ® 250 HHX (Hydroxyethyl Cellulose) | 45 |
| Copovidone | 22 |

A premix is prepared by passing guaifenesin through a milling machine into a V-shell mixer, adding the SYLOID® 244 silicon dioxide to the V-shell mixer, and mixing the guaifenesin with the silicon dioxide for five minutes. Thereafter, the dicalcium phosphate anhydrous, NATROSOL® 250 HHX hydrophilic polymer (hydroxyethyl cellulose) and compressible sugar are added to the V-shell mixer and mixed for 10 minutes with the guaifenesin and silicon dioxide. The resulting premix is combined with an aqueous copovidone solution and granulated in a granular to obtain a wet granulation. The wet granulation is placed in a fluid bed drier and dried to a moisture content to about 0.5-2.0% at 80° C. for 10 minutes. The dried granulation is milled (in a Comil milling machine).

TABLET FORMULATION

| Name of Ingredient | Mg/Tablet |
|---|---|
| Guaifenesin granulation | 752 |
| SYLOID ® 244 (Silicon Dioxide) | 17 |
| Compressible Sugar | 25 |
| Zinc Stearate | 5 |

The guaifenesin granulation is transferred to a V-blender, and the additional SYLOID® 244 (silicon dioxide) and compressible sugar are added to mix for 15 minutes. Thereafter, the zinc stearate is passed through a 30-mesh screen and added to the V-blender, and mixed with the granulation, and extragranular compressible sugar and silicon dioxide for five minutes.

The resulting mixture is compressed to obtain a 752 milligram tablet containing 600 milligrams of guaifenesin.

### EXAMPLE 4

A guaifenesin granulation comprising both intergranular and extragranular water-soluble and water-insoluble channeling agents is prepared in accordance with the following formulation.

GRANULATION

| Name of Ingredient | Mg/Tablet |
|---|---|
| Guaifenesin, USP | 600 |
| SYLOID ® 244 (Silicon Dioxide) | 12 |
| Dicalcium Phosphate Anhydrous | 3 |
| Compressible Sugar | 10 |
| NATROSOL ® 250 HX (Hydroxyethyl Cellulose) | 40 |
| Copovidone | 22 |

A premix is prepared by passing guaifenesin through a milling machine into a V-shell mixer, adding the SYLOID® 244 silicon dioxide to the V-shell mixer, and mixing the guaifenesin with the silicon dioxide for five minutes. Thereafter, the dicalcium phosphate anhydrous, NATROSOL® 250 HX hydrophilic polymer (hydroxyethyl cellulose) and compressible sugar are added to the V-shell mixer and mixed for 10 minutes with the guaifenesin and silicon dioxide. The resulting premix is combined with an aqueous copovidone solution and granulated in a granular to obtain a wet granulation. The wet granulation is placed in a fluid bed drier and dried to a moisture content to about 0.5-2.0% at 80° C. for 10 minutes. The dried granulation is milled (in a Comil milling machine).

TABLET FORMULATION

| Name of Ingredient | Mg/Tablet |
|---|---|
| Guaifenesin granulation | 687 |
| SYLOID ® 244 (Silicon Dioxide) | 17 |
| Compressible Sugar | 27 |
| Zinc Stearate | 5 |

The guaifenesin granulation is transferred to a V-blender, and the additional SYLOID® 244 (silicon dioxide) and compressible sugar are added to mix for 15 minutes. Thereafter, the zinc stearate is passed through a 30-mesh screen and added to the V-blender, and mixed with the granulation, and extragranular compressible sugar and silicon dioxide for five minutes.

The resulting mixture is compressed to obtain a 736 milligram tablet containing 600 milligrams of guaifenesin.

The above description is considered that of the preferred embodiments only. Modifications of the invention will occur to those skilled in the art and to those who make or use the invention. Therefore, it is understood that the embodiments shown and described above are merely for illustrative purposes and not intended to limit the scope of the invention, which is defined by the following claims as interpreted according to the principles of patent law, including the doctrine of equivalents.

The invention claimed is:

1. A pharmaceutical granulation, comprising:
a pharmaceutically active compound;
a hydrophilic polymer; and
a water-insoluble, non-swellable particulate channeling agent in an amount of at least 0.1 percent by weight.

2. The granulation of claim 1, wherein the pharmaceutically active compound is guaifenesin.

3. The granulation of claim 1, wherein the hydrophilic polymer is hydroxyethyl cellulose.

4. The granulation of claim 1, wherein the channeling agent is silicon dioxide.

5. The granulation of claim 1, wherein the hydrophilic polymer is present in an amount of from 3 to 30 percent by weight.

6. The granulation of claim 1 further comprising a non-hygroscopic diluent.

7. The granulation of claim 6, wherein the non-hygroscopic diluent is dicalcium phosphate anhydrous.

8. The granulation of claim 1, wherein the non-hygroscopic diluent is dicalcium phosphate anhydrous and the channeling agent is silicon dioxide.

9. The granulation of claim 1, further comprising a polymeric binder.

10. The granulation of claim 9, wherein the water-soluble channeling agent is compressible sugar.

11. The granulation of claim 1, further comprising a polymeric binder in an amount of from 0.5 to 5 percent by weight.

12. The granulation of claim 1, further comprising copovidone in an amount of from 0.5 to 5 percent by weight.

13. A pharmaceutical granulation comprising:
guaifenesin;
a hydrophilic polymer; and
a water-insoluble, non-swellable particulate channeling agent.

14. The granulation of claim 13, wherein the channeling agent is present in an amount of from 0.1 to 4.0 percent by weight.

15. The granulation of claim 13, wherein the hydrophilic polymer is hydroxyethyl cellulose.

16. The granulation of claim 13, wherein the channeling agent is silicon dioxide.

17. The granulation of claim 13, wherein the hydrophilic polymer is present in an amount of from 3 to 30 percent by weight.

18. The granulation of claim 13, wherein the granulation further comprises a non-hygroscopic diluent.

19. The granulation of claim 13, further comprising dicalcium phosphate anhydrous.

20. The granulation of claim 13, further comprising a polymeric binder.

21. The granulation of claim 13, further comprising a polymeric binder in an amount of from 0.5 to 5 percent by weight.

22. The granulation of claim 13, further comprising copovidone in an amount of from 0.5 to 5 percent by weight.

23. A process for preparing a pharmaceutical granulation, comprising:
blending a pharmaceutically active compound with a water-insoluble, non-swellable particulate channeling agent to obtain a first mixture;
adding a hydrophilic polymer, polymeric binder and any optional diluents and/or excipients to the first mixture, and blending to obtain a second mixture; and
wet granulating the second mixture to produce granules.

24. The process of claim 23, wherein the pharmaceutically active compound is guaifenesin.

25. The process of claim 23, wherein the hydrophilic polymer is hydroxyethyl cellulose.

26. The process of claim 23, wherein the channeling agent is silicon dioxide.

27. The process of claim 23, wherein the hydrophilic polymer is present in an amount of from 3 to 30 percent by weight.

28. The process of claim 23, wherein the granulation further comprises a non-hygroscopic diluent.

29. The process of claim 23, further comprising dicalcium phosphate anhydrous.

30. The process of claim 23, further comprising a polymeric binder.

31. The granulation of claim 23, further comprising a polymeric binder in an amount of from 0.5 to 5 percent by weight.

32. The process of claim 23, further comprising copovidone in an amount of from 0.5 to 5 percent by weight.

33. A compressed tablet or compressed tablet layer or portion comprising:
a therapeutically effective amount of a granulation including a pharmaceutically active compound, a hydrophilic polymer, and a water-insoluble, non-swellable particulate channeling agent in an amount of from 0.1 to 4.0 percent by weight of the granulation; and
one or more extragranular excipients, including a channeling agent.

34. The compressed tablet or compressed tablet layer or portion of claim 33, and one or more additional compressed tablet layers or portions.

35. A compressed guaifenesin tablet comprising:
a therapeutically effective amount of a granulation consisting essentially of guaifenesin, hydroxyethyl cellulose in an amount of from 3 to 30 percent by weight of the granulation, silicon dioxide in an amount of from 0.1 to 4.0 percent by weight of the granulation, copovidone in an amount of from 0.5 to 5 percent by weight of the granulation, dicalcium phosphate anhydrous in an amount up to 25 percent by weight of the granulation, and a compressible sugar in an amount up to 10 percent by weight of the granulation; and
one or more extragranular excipients, including a binder.
